SEQUENCE LISTING (10 sequences)

SEQ ID NO: 1 - protein, 8 aa, Artificial Sequence
  Cleavage Site for PreScission Protease
LEVLFQGP

SEQ ID NO: 2 - protein, 12 aa, Artificial Sequence
  Peptide Substrate
EAIYAAPFAKKK

SEQ ID NO: 3 - DNA, 64 bp, Artificial Sequence
  Primer
aaaagaaaaaaaggcggccgctcggatctgatcgaaggtcgttgtgcgggcaacttcgactcgg

SEQ ID NO: 4 - DNA, 40 bp, Artificial Sequence
  Primer
gcaaactggctcttccgcagccgctgaagtcctcatcggg

SEQ ID NO: 5 - protein, 18 aa, Artificial Sequence
  Xa-Cys-(Crk-II)-Intein-CBD Construct
MASSRVDGGRSDLIEGRC

SEQ ID NO: 6 - protein, 18 aa, Artificial Sequence
  Cys-F1-PS-Biotin Construct
CGXGLEVLFQGPVRKGXG

SEQ ID NO: 7 - protein, 11 aa, Artificial Sequence
  High affinity ligand for the N-SH3 Domain of Crk
PPPALPPKRRR

SEQ ID NO: 8 - protein, 318 aa, Artificial Sequence
  Protein Kinase Target
KRGCAGNFDSEERSSWYWGRLSRQEAVALLQGQRHGVFLVRDSSTSPGDYVLSVSENSRVSHYIINSSGPRPPVPPSPAQPPPGVSPSRLRIGDQEFDSLPALLEFYKIHYLDTTTLIEPVARSRQGSGVILRQEEAEYVRALFDFNGNDEEDLPFKKGDILRIRDKPEEQWWNAEDSEGKRGMIPVPYVEKYRPASASVSALIGGNQEGSHPQPLGGPEPGPYAQPSVNTPLPNLQNGPIYARVIQKRVPNAYDKTALALEVGELVKVTKINVSGQWEGECNGKRGHFPFTHVRLLDQQNPDEDFSGCGXGLEVLFQ

SEQ ID NO: 9 - protein, 326 aa, Artificial Sequence
  Recombinant Intermediate
KRGCAGNFDSEERSSWYWGRLSRQEAVALLQGQRHGVFLVRDSSTSPGDYVLSVSENSRVSHYIINSSGPRPPVPPSPAQPPPGVSPSRLRIGDQEFDSLPALLEFYKIHYLDTTTLIEPVARSRQGSGVILRQEEAEYVRALFDFNGNDEEDLPFKKGDILRIRDKPEEQWWNAEDSEGKRGMIPVPYVEKYRPASASVSALIGGNQEGSHPQPLGGPEPGPYAQPSVNTPLPNLQNGPIYARVIQKRVPNAYDKTALALEVGELVKVTKINVSGQWEGECNGKRGHFPFTHVRLLDQQNPDEDFSGCGXGLEVLFQGPVRKGXG

SEQ ID NO: 10 - protein, 5 aa, Artificial Sequence
  Site for Sequential Ligation
IEGRX

(19) United States
(12) Patent Application Publication
(10) Pub. No.: US 2006/0040319 A1
(43) Pub. Date: Feb. 23, 2006

(54) Multiple sensor-containing active modified polypeptides, preparation and uses thereof

(76) Inventors: Tom Muir, New York, NY (US); Graham Cotton, New York, NY (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/784,721

(22) Filed: Feb. 23, 2004

Related U.S. Application Data
(63) Continuation of application No. 09/483,543, filed on Jan. 14, 2000, now abandoned.

(51) Int. Cl. G01N 33/53

(52) U.S. Cl. 435/7.1

(57) ABSTRACT

Compositions and methods are provided for identifying conformational changes in polypeptides related to the activity or biological state of the polypeptide. Semisynthetic polypeptides are prepared comprising at least two proximity-sensor peptides, the resultant composition capable of detectably indicating the activity of biological state of the polypeptide. Such compositions may be used to identify modulators of the polypeptides as well as modulators of molecules which interact with the polypeptide, such as protein kinases which act on protein kinase targets.

### GOVERNMENTAL SUPPORT

The research leading to the present invention was supported, at least in part, by a grant from the National Institutes of Health (GM55843-01). Accordingly, the Government may have certain rights in the invention.

### FIELD OF THE INVENTION

The invention relates to semisynthetic polypeptides bearing at least two sensors which report the relative configuration of the polypeptide and its activity or biological state, and methods of their use, for monitoring activity, identifying modulators of activity, and identifying agents capable of altering the activity of the modulators

### BACKGROUND OF THE INVENTION

The incorporation of biophysical probes or post-translational modifications at defined positions within a target protein, provides an extremely powerful way of investigating the molecular mechanisms which control complex biological processes. There are now several methods available for labeling a recombinant protein at a single defined position, in particular, unnatural amino acid mutagenesis (1) and cysteine modification (e.g. (2)) have been extensively used for this purpose. However, these approaches do not offer a straightforward way of introducing multiple different modifications at specific sites within a protein in a homogeneous fashion. Thus, sophisticated protein engineering strategies which require specific combinations of biophysical/biochemical probes to be incorporated into proteins, e.g. fluorescence resonance energy transfer (FRET) pairs, isotopic labels and post-translational modifications, have proven extremely difficult to perform. In principle, protein total synthesis via the chemical (3) or enzymatic (4) ligation of synthetic peptide fragments provides a route to proteins possessing diverse patterns of chemical modification. Although these peptide ligation approaches have proven extremely powerful for studying small proteins (3), their practical utility decreases with increasing size of the target protein due to size constraints on the synthetic peptide building blocks (5).

Methods for chemically ligating two oligopeptides end to end with an amide bond, wherein at least one of the oligopeptides is a product of recombinant expression, have been described in U.S. patent Ser. No. 08/969,909, filed Nov. 13, 1997; Ser. No. 09/122,312, filed Jul. 24, 1998; Ser. No. 09/191,605, filed Nov. 13, 1998; and Ser. No. 09/191,890, filed Nov. 13, 1998, all of the foregoing incorporated herein by reference in their entireties.

Random incorporation of multiple sensor probes has been performed (e.g., (36)), but such non-specific dual labeling does not provide the selectivity nor specificity for providing useful monitoring of conformational changes generally, nor the sensitivity for specific proteins.

It is towards the facile preparation of multiple sensor labeled polypeptides capable of reporting conformational changes, and uses of the labeled polypeptides for identifying modulators of activities which induce such conformational changes, that the present invention is directed.

The citation of any reference herein should not be construed as an admission that such reference is available as “Prior Art” to the instant application.

### SUMMARY OF THE INVENTION

In one aspect, the present invention is directed to a semisynthetic composition comprising a preselected polypeptide incorporating at least two sensor peptides which detect and report changes in their relative proximities. The sensor peptides are located in the amino acid backbone, and the relative proximities of the peptides are capable of changing in relation to the activity or biological state of the polypeptide. Non-limiting examples of activities in which the polypeptide may participate and which results in reported changes by a composition of the invention include intramolecular interactions, intermolecular interactions, interaction with a ligand, interaction with a substrate, change in dielectric constant, change in pH, change in protein folding, post-translational modification, or modification of a residue. A preferred activity is phosphorylation and dephosphorylation, and the preselected polypeptide is a protein kinase or a protein kinase substrate. In a more preferred example, a protein kinase substrate is Crk-II.

By way of example, the composition of the invention may have a first proximity-sensor peptide at the N-terminus, the C-terminus of which is peptide-bonded to the N-terminus of the recombinant portion, the C-terminus of which is peptide bonded to the N-terminus of a second interacting proximity-sensor peptide. The recombinant portion comprises the part of the preselected polypeptide which undergoes the conformational change in relation to activity. In a preferred embodiment, the recombinant portion has an N-terminal cysteine and a C-terminal ^{α}thioester. The at least two interacting proximity-sensor peptides are capable of detectably communicating their relative proximities and changes thereto. By way of example, the at least two interacting proximity-sensor peptides comprise a FRET pair. Examples of FRET pairs include but are not limited to fluorescein and tetramethylrhodamine, IAEDANS and fluorescein, EDANS and DABCYL, BODIPY FL fluorescein and BODIPY fluorescein, P-phycoerythrin and CY5, and pyrene and coumarin.

By way of example, a composition of the invention is depicted in FIG. 5A (SEQ ID No:8).

The composition of the invention may have a third interacting proximity-sensor peptide.

In another aspect, the invention is directed to a method for measuring changes in the relative proximity between at least a first position and a second position in a preselected polypeptide, the polypeptide capable of participating in an activity, the changes related to the activity of the polypeptide, the method comprising the steps of:
(a) preparing the composition as described hereinabove;
(b) subjecting the composition to conditions inducing the activity; and
(c) measuring the changes in relative proximity of the first and second interacting proximity-sensor peptides in the composition.

Conditions capable of inducing the activity include interaction of the composition with a substrate, interaction of the composition with a ligand, interaction of the composition with a binding partner, interaction of the composition with an enzyme, post-translational modification, change in pH, change in dielectric constant, and change in protein folding. Means for measuring the changes is performed by a method such as but not limited to fluorescence spectroscopy, nuclear magnetic resonance spectroscopy, electron spin resonance spectroscopy, ultraviolet/visible spectroscopy, and extent of cross-linking by cross-linking agents.

In another aspect of the invention, a method is provided for identifying an agent capable of modulating the activity or biological state of a preselected polypeptide, or for identifying an agent capable of affecting the activity of a modulator of the activity or biological state of the polypeptide, the activity detectable by changes in the relative proximity among at least a first position and at least a second position in the preselected polypeptide. Thus, for example, agents may be identified which alter the activity or biological state of the composition directly, or, in another and preferred embodiment, agents may be identified which alter the activity of an enzyme or other molecule which acts on the composition of the invention. These methods are achieved by the steps of:
(a) providing the composition as described hereinabove;
(b) subjecting the composition to conditions inducing the activity in the presence and absence of the agent;
(c) measuring the changes in relative proximity of the first and second interacting proximity-sensor peptides in the composition in the presence and absence of the agent; and
(d) identifying the agent affecting the changes as capable of modulating the activity or modulating the modulator of the activity.

The activity may be a consequence of, for example, intramolecular interactions, intermolecular interactions, interaction with a ligand, interaction with a substrate, change in dielectric constant, change in pH, change in protein folding, post-translational modification, or modification of a residue. In a preferred embodiment, the post-translational modification is phosphorylation and dephosphorylation. The preselected polypeptide may be a protein kinase substrate. A preferred protein kinase substrate is Crk-II.

In the foregoing example, if the activity to be measured is the effect of an agent on a molecule which modulates the activity of the composition, the conditions of the method will include the composition, the molecule which acts on the composition to modulate its activity, and other reagents or other factors necessary for the activity to occur. The measurements are then also made in the presence of a candidate agent which may affect the molecule. By way of specific but non-limiting example to illustrate this aspect of the invention, the composition is a dual-labeled, semisynthetic polypeptide comprising the protein kinase adapter protein Crk-II capable of reporting phosphorylation. The molecule modulating its activity is the protein kinase c-Abl. Other factors to permit phosphorylation of the composition by c-Abl are present. Candidate inhibitor agents of c-Abl activity may be added and the effect of the agent on phosphorylation of the composition monitored by the reporting of the proximity of the sensors in the composition. Under normal conditions, the composition will be phosphorylated, the change in conformation of the composition detected by fluorescence changes in the FRET pair. Inclusion of an agent which inhibits the protein kinase activity will be detected by an alteration in the expected fluorescence changes during phosphorylation. Both inhibitors and activators of the protein kinase activity may be identified by these methods. These principles of the invention apply to identifying antagonists and agonists of other interacting molecules, in which one is provided as a labeled composition as embraced by the invention herein.

Thus, in a specific embodiment, the activity is phosphorylation, the method comprising providing a semisynthetic target of phosphorylation activity capable of reporting phosphorylation activity, providing a protein kinase capable of phosphorylating the target, providing candidate modulators of the activity of the protein kinase, and determining the effectiveness of the modulators of the protein kinase activity by measuring the reported activity of the target. In a preferred example, the semisynthetic reported target is a modified Crk-II, the protein kinase is c-Abl, and the modulators are agonists or antagonists of c-Abl activity.

In a preferred embodiment, the first interacting proximity-sensor peptide is at the N-terminus, the C-terminus of which is peptide-bonded to the N-terminus of the recombinant portion, the C-terminus of which is peptide bonded to the N-terminus of the second interacting proximity-sensor peptide. The recombinant portion may have an N-terminal cysteine and a C-terminal ^{α}thioester. The at least two interacting proximity-sensor peptides are capable of detectably communicating their relative proximities and changes thereto. In a preferred embodiment, the at least two interacting proximity-sensor peptides comprise a FRET pair. The interacting proximity-sensor peptide may be a synthetic oligopeptide comprising a fluorescent amino acid derivative. In a preferred embodiment, the fluorescent amino acid derivative comprises a fluorophore selected from the group consisting of fluorescein, tetramethyl rhodamine, EDANS, IAEDANS, DABCYL, BODIPY fluorescein, β-phycoerythrin, CY5, pyrene, or coumarin. Appropriate pairs of fluorophores to act as a FRET pair will be readily selected by the skilled artisan.

In a preferred aspect of the invention, a method is provided for measuring changes in the relative proximity between at least a first position and a second position in Crk-II, these changes related to the activity of Crk-II, comprising the steps of:
(a) providing a modified, dual-labeled Crk II molecule such as SEQ ID No:8;
(b) subjecting said composition to conditions inducing activity; and
(c) measuring the changes in relative proximity of the first and second interacting proximity-sensor peptides in the composition.

Preferred conditions inducing the activity is phosphorylation and dephosphorylation; measuring the changes is performed by fluorescence spectroscopy. The phosphorylation and dephosphorylation may be induced by c-Abl or the epidermal growth factor receptor.

In yet another aspect of the intention, a method is provided for identifying an agent capable of modulating the activity of a protein kinase by measuring changes in the relative proximity among at least a first position and at least a second position in a modified, dual-labeled modified protein kinase target (adapter) protein, such as Crk-II, comprising the steps of:
(a) providing a modified, dual-labeled protein kinase target (adapter) protein;
(b) subjecting the dual-labeled protein kinase target (adapter) protein to conditions wherein it is acted on upon a protein kinase, in the presence and absence of a candidate agent;
(c) measuring the changes in relative proximity of the first and second interacting proximity-sensor peptides in the target protein in the presence and absence of the agent; and
(d) identifying an effective agent as one capable of modulating the activity of the protein kinase.

In a preferred embodiment, the protein kinase is c-Abl, the target (adapter) protein is Crk-II, and the modified, dual-labeled protein kinase target is the structure depicted in FIG. 5A (SEQ ID No:8).

In yet another aspect of the intention, a method is provided for identifying an agent capable of modulating the activity of a protein kinase capable of phosphorylating Crk-II, by changes in the relative proximity among at least a first position and at least a second position in a modified, dial-labeled modified Crk-II polypeptide, comprising the steps of:
(a) providing a modified, dual-labeled Crk II molecule such as the Rh-(Crk-II)-Fl construct of FIG. 5A and SEQ ID No:8;
(b) subjecting the dual-labeled molecule to conditions inducing the activity in the presence and absence of the agent;
(c) measuring the changes in relative proximity of the first and second interacting proximity-sensor peptides in the composition in the presence and absence of the agent; and
(d) identifying an agent affecting the changes as capable of modulating the activity.

In still a further embodiment of the invention, a method is provided for preparing a composition comprising a preselected polypeptide capable of communicating changes in the relative proximity among at least one first position and at least one second position in the preselected polypeptide, the changes related to the activity of the preselected polypeptide, comprising the steps of:
(a) providing at least a first interacting proximity-sensor peptide and a second interacting proximity-sensor peptide, each of the peptides having an interacting proximity-sensitive moiety present therein, the moieties capable of communicating changes in their relative proximities;
(b) providing at least one recombinant polypeptide or portion of said preselected polypeptide, the recombinant portion having an N-terminal cysteine, a C-terminal ^{α}thioester, or the combination thereof;
(c) ligating the at least one recombinant polypeptide or portion thereof and the at least first and second interacting proximity-sensor peptides into an amino acid backbone at the first position and at least one second position to provide a composition comprising the preselected polypeptide, such that in the composition the relative proximities of the positions of the second interacting proximity-sensor peptides are capable of changing in relation to the activity of the composition.

These and other aspects of the present invention will be better appreciated by reference to the following drawings and Detailed Description.

### BRIEF DESCRIPTION OF THE DRAWINGS

FIG. 1. Biosensor for c-Abl phosphorylation of the Crk-II adapter protein. c-Abl phosphorylates Crk-II on Tyr 221 which is thought to induce an intramolecular association with the SH2 domain. This rearrangement is expected to yield a net change in the distance between the termini of the protein, which would be reported by a dual-labeled derivative of Crk-II in which the FRET pair tetramethylrhodamine (Rh) and fluorescein (Fl) are specifically incorporated at its - and C-termini, respectively.

FIG. 2. Solid-phase protein ligation (SPPL). (A) Generation of Rh-(Crk-II)-Fl (SEQ ID No:8). Analogous to SPPS, the procedure involves a loading step followed by rounds of deprotection and ligation, and culminates in a cleavage step. Av, monomeric avidin; Bio, biotin. (B) Coomassie-stained 12% SDS-PAGE gel of: lane 1, molecular weight markers; lane 2, monomeric avidin beads loaded with the first ligation product Xa-Cys-(Crk-II)-Fl-PS-Biotin; lane 3, the same beads after treatment with factor Xa to yield Cys-(Crk-II)-Fl-PS-Biotin; lane 4, the beads after overnight ligation of the second synthetic peptide to generate Rh-(Crk-II)-Fl. (C) ESMS (expected mass=37,123.6 Da) and (D) fluorescence emission spectrum (excitation 490 nm) of purified Rh-(Crk-II)-Fl.

FIG. 3. Phosphorylation of Rh-(Crk-II)-Fl by full length c-Abl. Rh-(Crk-II)-Fl was treated with recombinant c-Abl with or without ATP. (A) The percentage change in the Fl:Rh fluorescence emission intensity ratio of Rh-(Crk-II)-Fl at 1 min. and 60 min. time points. (B) Anti-phosphotyrosine western analysis of the corresponding Rh-(Crk-II)-Fl samples shown in (A). (C) 6% Native-PAGE gel of untreated Rh-(Crk-II)-Fl (lane 1), Rh-(Crk-II)-Fl after treatment with c-Abl for 60 min in the absence of ATP (lane 2), and Rh-(Crk-II)-Fl after treatment with c-Abl for 60 min in the presence of ATP (lane 3). The gel was imaged for fluorescein fluorescence using a Storm instrument (Molecular Dynamics). All experiments were performed in triplicate.

FIG. 4. (A) Change in Rh-(Crk-II)-Fl fluorescence after treatment with a truncated version of c-Abl containing only the SH2 and kinase domains. Kinase reactions were performed over 60 min. with or without the addition of ATP. Anti-phosphotyrosine western analysis of the corresponding Rh-(Crk-II)-Fl samples shown below. As a positive control, an equimolar amount of Rh-(Crk-II)-Fl was treated with full length c-Abl and ATP for 60 min. (B) Change in Rh-(Crk-II)-Fl fluorescence after treatment with full length c-Abl in the presence of a saturating concentration of a high affinity peptide ligand for the N-SH3 domain of Crk-II. As above, the anti-phosphotyrosine western analysis of the respective reactions is shown directly below the fluorescence data. In both FIGS. A and B the fluorescence values are the mean over three measurements.

FIG. 5 depicts the structure of (A) a dual-labeled, semisynthetic, recombinantly-prepared composition comprising the protein kinase adapter protein Crk-II which is capable of reporting phosphorylation by c-Abl; and (B) a recombinant intermediate in the preparation of (A) above. Dapa(Fl) refers to diaminopropionic acid-fluorescein, and Rh refers to tetramethylrhodamine.

### DETAILED DESCRIPTION OF THE INVENTION

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Therefore, if appearing herein, the following terms shall have the definitions set out below.

In keeping with standard polypeptide nomenclature, J. Biol. Chem., 243:3552-59 (1969), abbreviations for amino acid residues are shown in the following Table of Correspondence:

TABLE OF CORRESPONDENCE

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| W | Trp | tryptophan |
| R | Arg | arginine |
| D | Asp | aspartic acid |
| N | Asn | asparagine |
| C | Cys | cysteine |

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues. The above Table is presented to correlate the three-letter and one-letter notations which may appear alternately herein.

A “replicon” is any genetic element (e.g., plasmid, chromosome, virus) that functions as an autonomous unit of DNA replication in vivo; i.e., capable of replication under its own control.

A “vector” is a replicon, such as plasmid, phage or cosmid, to which another DNA segment may be attached so as to bring about the replication of the attached segment.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

An “origin of replication” refers to those DNA sequences that participate in DNA synthesis.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

The term “oligonucleotide,” as used herein in referring to the probe of the present invention, is defined as a molecule comprised of two or more ribonucleotides, preferably more than three. Its exact size will depend upon many factors which, in turn, depend upon the ultimate function and use of the oligonucleotide.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

As used herein, the terms “restriction endonucleases” and “restriction enzymes” refer to bacterial enzymes, each of which cut double-stranded DNA at or near a specific nucleotide sequence.

FRET or fluorescence resonance energy transfer is a distance-dependent interaction between the electronic excited states of two (or more) dye molecules in which excitation is transferred from a donor molecule to an acceptor molecule without emission of a photon.

A proximity-sensor peptide refers to a peptide comprising a moiety capable of reporting its proximity on interacting with another peptide comprising a moiety, the moieties being, for example, a FRET pair.

An object of the present invention is to provide a generally accessible methodology which allows several recombinant and synthetic polypeptides to be regioselectively linked together, thereby allowing multiple different chemical probes to be site-specifically incorporated into the resulting semi-synthetic protein product. Proteins undergo conformational changes related to their activity or modified state, such as protein targets of phosphorylation and dephosphorylation. By use of probes which are environmentally sensitive, for example, those which are proximity sensitive, changes in their interaction may be monitored to identify the activity or state of the polypeptide. Thus, with a target of biological activity capable of reporting its activity and facile detection of the activity, the target is useful for several purposes. One such purpose is in identifying modulators of the interaction between the target and a molecule which affects the activity or biological state of the target. By way of non-limiting example, which will be exemplified in the Examples below, agents capable of modulating protein kinase activity may be identified using the constructs and methods herein. For example, a protein kinase and its target protein, the latter provided as a semisynthetic construct of the invention labeled to report the state and kinetics of phosphorylation is used. Under appropriate conditions, the combination of the protein kinase and the labeled target reports the protein kinase activity. By carrying out the measurement of the protein kinase activity in this manner in the presence and absence of a candidate agent for modulating protein kinase activity, one may identify inhibitors or activators of the protein kinase. Moreover, the inhibitors or activators may act on the protein kinase, or on the substrate, or both; further studies may be preformed to identify the site of interaction. Agents capable of modulating the kinetics of enzymatic activity are detectable using these methods.

While the foregoing example is merely illustrative of the broad utility of the invention, other targets, other modulators of the targets, and agents or other conditions capable of further modulating the modulators of the targets are candidates for study and screening as disclosed herein. Interactions that may be investigated using the methods of the invention include those between enzymes and their substrates (even if the substrates are themselves enzymes), receptors with ligands, other intramolecular interactions, intermolecular interactions, other interactions with ligands, other interactions with a substrate, effects of changes in dielectric constant, effects of changes in change in pH, effects of change in protein folding, post-translational modification, or modification of a residue. As mentioned above, in a preferred embodiment, the post-translational modification is phosphorylation and dephosphorylation. The preselected polypeptide may be a protein kinase substrate. A preferred protein kinase substrate is Crk-II; the protein kinase being c-Abl. As noted above, the methods herein can be used to monitor effects on reaction rate, turnover, extent of reaction, and other aspects of interactions between interacting molecules.

Of course, active fragments of the preselected polypeptide (target) capable of being acted on and reporting activity may be used in the composition and methods herein, as well as active fragments of molecules capable of modulating the activity of the target. With regard to the latter, the biological activity of, for example, an enzyme, may be studied using a target composition of the invention as its substrate, wherein the activity of fragments of the enzyme may be determined by its activity directed to a target composition of the invention. In general, the compositions of the invention as targets or as substrates for various biological phenomena may be utilized to evaluate all aspects of the interaction of the substrate with a molecule or molecules directly or indirectly affecting the activity of the molecule of the substrate.

This facile approach to identifying modulators of activity offers significant improvements over previous screening methods, for example the methods of the invention may be performed without the need for radioactively labeled materials. By way of example, protein kinase activity may be measured by the extent of phosphorylation of protein kinase targets using a ³²P-labeled substrate and the incorporation of ³²P-phosphate into the targets. Such methods require expensive and potentially hazardous radioactive materials, means for the safe handling and disposal of such materials, and associated instrumentation for measuring radioactivity. In contrast, by use of the methods of the invention, conformational changes upon phosphorylation and dephosphorylation of the target protein are easily detected, for example by fluorescence spectroscopy, readily allowing the effect of agents capable of modulating the activity to be measured. As mentioned previously, such agents may interact with the phosphorylation target, the enzyme, or auxiliary or other proteins or other factors which modulate phosphorylation/dephosphorylation.

The multiple labeled polypeptides of the invention used in the methods are readily prepared by various methods; one preferred but non-limiting method is by sequential peptide ligation, an iterative fragment condensation strategy which allows a series of unprotected peptide building blocks to be assembled in a unidirectional, stepwise fashion. Such building blocks in the examples herein include the target protein or a fragment thereof and peptides which comprise the probes. The so-called ‘native chemical ligation’ reaction was chosen as the synthetic framework for the approach since it allows two peptide fragments to be joined together via a normal peptide bond (6), and because it has been successfully applied to the sequential ligation of multiple synthetic peptides both in solution (7, 8) and, most recently, on the solid-phase (9). Importantly, recent advances in protein engineering allow the necessary reactive functionalities for native chemical ligation (namely, an N-terminal cysteine and a C-terminal thioester) to be introduced into recombinant polypeptides (10-13). This has enabled semi-synthetic and fully recombinant protein constructs to be generated through ligation of the appropriate two fragments, in a procedure termed expressed protein ligation (EPL) (14) or intein-mediated protein ligation (15) (for review see (16)). This method is merely illustrative of a preferred embodiment of the invention; other methods may be used to prepare the desired multiple-labeled polypeptides of the invention.

EPL has been extended to permit the insertion of a synthetic peptide into a recombinant protein through the sequential ligation in solution of two recombinant protein fragments to the - and C-termini of a synthetic peptide cassette (17). While this strategy is, theoretically, extendible to the ligation of any combination of synthetic and/or recombinant fragments, the need to perform all of the steps in solution renders the approach technically demanding; after each ligation reaction it is necessary to isolate the desired product from the reaction mixture, a process which is time-consuming and, importantly, leads to substantial handling losses. In principle, these problems should be overcome by transferring the entire process to the solid-phase, in a manner analogous to solid-phase peptide synthesis (SPPS) (18). As with SPPS, this solid-phase protein ligation (SPPL) approach should allow each reaction to be driven to completion by using a large excess of reagents, which can then be simply removed by washing. In addition, there would be no need to isolate intermediate ligation products which would remain immobilized on the support. The present inventors have developed an SPPL technology and have successfully applied it to the generation of a dual-labeled version of the ˜35 kDa adapter protein, Crk-II. As is shown herein, this semi-synthetic protein analog specifically biosenses a post-translational tyrosine phosphorylation event important in regulation of Crk-II mediated signal transduction. Thus, it may be used for various purposes, such as to identify agents capable of modulating phosphorylation activity. It is also only an example of other protein kinase targets, and more broadly, other useful polypeptides that biosensing conformational changes therein is useful in screening and other purposes, as noted below.

Various polypeptides which undergo conformational changes upon post-translational modification or other effects are candidates for the preparation of a semi-synthetic multiple labeled polypeptide constructs of the invention. Proteins which are themselves targets of enzymatic modification are preferred examples; targets of protein kinase activity are particularly preferred. Non-limiting examples of such targets include transcription factors and signal transduction factors. Numerous other targets are embraced herein, such as those reviewed in (35). In a most preferred embodiment, the polypeptide is an adapter protein. In a more preferred embodiment, the target is a target of the protein kinase c-Abl, such as Crk-II. FIG. 1 illustrates the conformational change which the adapter protein undergoes on phosphorylation, and the change in proximity of a dual-labeled composition of the invention comprising the Crk-II polypeptide. The polypeptide of the invention may be comprise the sequence of the entire target protein, or may comprise a fragment of the sequence, the fragment which comprises the site of the post-translational modification and the portions of the polypeptide which undergo the conformational changes to be measured in an aspect of the instant invention. Various modifications which do not detract from the utility of the fragment may be made, for example, to facilitate ligation to the sensor peptides, expression, optimal placement of the sensor peptides, and ease of synthesis or purification, among others.

Two or more probes may be provided in the semi-synthetic polypeptide. Such probes are selected to report their relative proximities. For example, fluorescence resonance energy transfer (FRET) pairs provide a fluorescence reading depending on the proximity of the fluorophores. For example, fluorescein and tetramethylrhodamine may be used. Other pairs include IAEDANS and fluorescein, EDANS and DABCYL, BODIPY FL fluorescein and BODIPY fluorescein, β-phycoerythrin and CY5, and pyrene and coumarin. FRET pairs are known in the art and a skilled artisan can readily select appropriate pairs for use in the compositions of the invention. The probes of the invention are modified peptides in which the fluorophore or other reporter moiety is provided as a side chain or in the polypeptide backbone. Examples include Dapa-fluorescein (diaminopropionic acid-fluorescein) and N^{α}-tetramethylrhodamine-KRG. Others include peptides or oligopeptides with a moiety, such as EDANS, IAEDANS, DABCYL, BODIPY fluorescein, β-phycoerythrin, CY5, pyrene, or coumarin, capable of participating as a FRET pair with another modified oligopeptide. As noted in the examples herein, which are not limiting, the labeled peptides are provided in forms to be incorporated in a stepwise fashion into the dual-labeled polypeptide. In one synthetic strategy, as described in the Examples below, the labeled peptides may be provided in a form for eventual enzymatic of, chemical cleavage to, for example, release the product from a substrate. Thus, the reactants may have cleavage sites therein to facilitate synthesis. In an example herein, shown in FIG. 1, Crk II (adapter protein; phosphorylation target of c-Abl) is recombinantly expressed as a fusion construct at the N-terminus of an intein-chitin binding domain (Xa-Cys-(Crk-II)-Intein-CBD). An N-terminal cysteine is included to facilitate ligation. The recombinant construct is bound to chitin beads through the chitin-binding domain. In the first step, the above construct is reacted with CGK(Fl)-GLEVFQGPVRKGK(Biotin)GNH₂ (“Cys-Fl-PS-Biotin”; SEQ ID No:6), wherein the N-terminal cysteine is ligated to the Crk-II, forming the product Xa-Cys-(Crk-II)-Fl-PS-Biotin. The ligated product is then bound to avidin beads through the biotin moiety on the C-terminal portion of the fluorescein-labeled peptide. The Xa portion is then cleaved with factor Xa, and the now-exposed N-terminal cysteine reacted with N^{α}-tetramethylrhodamine-KRG-propionamide ^{α}thioester to ligate the cysteine with the thioester. Subsequently, the PS peptide is cleaved, yielding the dual-labeled product.

While the above stepwise reaction scheme forms the desired product, other means to prepare the multiple sensor labeled polypeptide may be carried out within the teachings of the present invention.

The present invention is directed to the semi-synthetic constructs comprising a target polypeptide and multiple probes, as well as methods for using these constructs in monitoring the biological activity of the polypeptide upon modification (or return to its native state) as well as its use in identifying agents capable of modulating the modification. Numerous examples of polypeptides that are targets of post-translational and other modifications, especially reversible modifications, are available. By way on non-limiting example, targets of protein kinase activity are preferred embodiments of the present invention. Such include signal transduction factors and transcription factors, as non-limiting examples, as further exemplified in (35). Protein kinases and their phosphorylation/dephosphorylation targets are implicated in critical pathways in which perturbations are known to lead to clinically significant derangements, such as cellular transformation and carcinogenesis. In particular, the protein kinase c-Abl and its target Crk-II are involved in cellular regulation, derangements of which can lead to cellular dysfunctions. Identification of molecules capable of preventing phosphorylation of Crk-II are candidates for pharmaceutical development. Heretofore, assays of compounds for modulation of phosphorylation required the use of ³²P and critical measurements of labelling of target molecules. The instant invention provides a facile means to identify modulators of phosphorylation by monitoring changes in the interaction of multiple labels on the phosphorylation target, induces by changes in confirmation consequent to phosphorylation. Rapid, automated high-throughput screening of compounds may be performed using the constructs and methods of the present invention.

The positions of the multiple probes in the final construct are selected to report changes in conformation of the construct. Thus, the positions may be situated wherever conformational changes occur. In the example herein, the probes are located on the N- and C-termini of the molecule, but this need not be the case for every labeled polypeptide. Such positions will be selected based on the known interactions and conformational changes in the molecule upon post-translational modification, and the polypeptide may be thus constructed. Therefore, the probes may be in the polypeptide chain at the ends, or bounding either side of the target sequence, but having an additional polypeptide bound thereto. By way of example, if P1 and P2 are the probes, and T is the polypeptide sequence containing the target site, and A and B are other intervening peptides, the following examples of constructs are embraced herein: P1-T-P2, A-P1-T-P2, A-P1-T-B-P2, P1-T-B-P2, P1-A-T-P2, P1-A-T-P2-B, P1-B-T-P2-A, A-P1-T-P2-B, and B-P1-T-P2-A. Of course, further constructs may be prepared without deviating from the spirit of the invention. The sensors may be placed at any suitable position at which conformational changes in the polypeptide alter the proximity of the sensors and result in a detectable change, or report, of the alteration. Such positions may be determined by study of the polypeptide, or by preparation and testing of the constructs of the invention.

As will be seen in the Examples below, the synthesis was carried out of a semi-synthetic version of the adapter protein, Crk-II, in which the FRET pair, tetramethylrhodamine and fluorescein were incorporated at the - and C-termini of the protein, respectively (hereafter referred to as Rh-(Crk-II)-FI), as described in summary above. Crk-II has been implicated in a number of cellular signaling processes, and is composed predominantly of one Src homology 2 (SH2) and two SH3 domains through which it mediates intermolecular protein-protein interactions (22, 23). Two protein tyrosine kinases, c-Abl and the epidermal growth factor receptor (EGFR), are known to phosphorylate Crk-II on a unique tyrosine residue (Tyr221) located between the SH3 domains (24, 25). This post-translational modification is thought to regulate Crk-II function by inducing an intramolecular association with the SH2 domain (26) which in turn inhibits certain intermolecular protein interactions (22-25). It was anticipated that phosphorylation and subsequent intramolecular association would result in a distance change between the termini of Crk-II, which would lead to a change in FRET between the two fluorophores in the dual-labeled analog (FIG. 1). Consequently, this protein construct would directly biosense this important post-translational event.

The preparation of the construct Rh-(Crk-II)-Fl is summarized in FIG. 2A. As with SPPS, the strategy can be divided essentially into three parts; attachment of the first building block to a solid support (e.g., avidin beads), chain assembly in a C-to-N direction involving successive deprotection and ligation steps, and cleavage of the completed polypeptide off the solid support. In the first step, full length mouse Crk-II was expressed as an in-frame fusion to an engineered yeast VMA intein which allows the subsequent generation of a reactive ^{α}thioester derivative of Crk-II. In this example, an extra Gly residue was added to the C-terminus of Crk-II to improve the kinetics of the first ligation reaction (8), and the N-terminal Met was replaced by the sequence -IEGRC (Xa-Cys) to facilitate controlled sequential ligation (17). Soluble expression of this fusion protein [Xa-Cys-(Crk-II)-Intein-CBD] was optimized using standard protocols (no in vivo intein cleavage of the full length fusion could be detected) and the desired material purified by affinity chromatography using a chitin column.

A synthetic peptide, Cys-Fl-PS-Biotin, containing both a fluorescein probe (Fl) and a biotin affinity handle separated by a linker region containing the cleavage site for the PreScission protease [LEVLFQGP (SEQ ID No:1), (PS)], was chemoselectively ligated to the C-terminus of recombinant Crk-II using EPL. This ligation reaction was found to be >95% complete after 48 h in the presence of a large excess of peptide and the thiol cofactors ethanethiol and MESNA. Gel filtration was used to separate the unreacted peptide from the desired ligation product which was then attached to monomeric-avidin beads via its biotin functionality. Preliminary model studies had established that the monomeric-avidin-biotin complex was stable to all the washing, deprotection and ligation steps used in SPPL, but that the interaction can be disrupted under mild conditions with exogenous biotin. Trace amounts of unreacted Crk-II protein and any remaining bacterial protein contaminants were then removed by vigorously washing the beads with high salt and detergent at pH 5.2 and pH 8.0. This yielded the pure protein, Xa-Cys-(Crk-II)-Fl-PS-Biotin, immobilized on a solid-support (FIG. 2B, Lane 2).

In order to continue the solid-phase synthesis, the Xa pro-sequence must be removed from the immobilized Xa-Cys-(Crk-II)-Fl-PS-Biotin to give an N-terminal Cys residue ready for ligation to the next peptide fragment. (The Xa motif acts as an N^{α} protecting group for the Cys residue in Crk-II and prevents uncontrolled self-ligation during the first ligation step (17)). Complete enzymatic deprotection was achieved by treatment of the beads with the protease, factor Xa, for 3 hours to give Cys-(Crk-II)-Fl-PS-Biotin (FIG. 2B, Lane 3). A small amount (˜10%) of a lower molecular weight protein contaminant was also observed (FIG. 2B, Lane 3, weak band ˜26 kDa) suggesting that some non-specific cleavage had occurred during this step. The proteolysis reaction was terminated by simply washing the protease from the column; DTT was included in this buffer to simultaneously reduce any disulfide bonds that may have formed during the deprotection step. The beads were then equilibrated into ligation buffer, and the newly exposed N-terminal cysteine residue reacted with a tetramethylrhodamine containing ^{α}thioester peptide (Rh-KRG-propionamide ^{α}thioester) in a second ligation step. A large excess of synthetic peptide was again used in the reaction and MESNA was added as the sole thiol cofactor. This reaction was deemed complete after overnight incubation, as determined by SDS-PAGE analysis of the beads (FIG. 2B, Lane 4), generating the dual-labeled Crk-II derivative, Rh-(Crk-II)-Fl. The beads was then thoroughly washed to remove all unreacted tetramethylrhodamine peptide.

Rh-(Crk-II)-Fl was desorbed from the solid support by washing the beads with a solution containing 2 mM biotin. Approximately 55% of the immobilized material was recovered in a single washing step, though further protein could be eluted by repeating this procedure. The combined washes were passed over a gel filtration column to remove the free biotin and to remove the protein contaminant arising from non-specific factor Xa proteolysis. The so-purified dual-labeled Crk-II analog was characterized by electrospray mass spectrometry (FIG. 2C) and fluorescence spectroscopy (FIG. 2D), and was shown to bind a phosphotyrosine column and a peptide ligand specific to the central SH3 domain of Crk-II, indicating that it had the same gross functional properties as the wild-type protein.

Tetrameric-avidin was used as the solid support for SPPL. However, due to the high affinity of this interaction, the completed protein cannot be competitively eluted from the column as above. In this case the beads were treated with the highly specific PreScission protease. The enzyme cleaved the construct at its recognition site, incorporated between the fluorescein and the biotinyl functionality's, releasing Rh-(Crk-II)-Fl from the beads.

As will be shown in a further Example, below, phosphorylation studies were performed on the construct to demonstrate its utility in identifying modulators of protein kinase activity. Purified Rh-(Crk-II)-Fl was assayed for its ability to biosense Crk-II phosphorylation by the c-Abl protein tyrosine kinase. As indicated previously, phosphorylation by c-Abl leads to an intramolecular association between a phosphotyrosine motif and the Crk-II SH2 domain, which can be reported by the dual-labeled Crk-II derivative (FIG. 1). Rh-(Crk-II)-Fl was treated with full length recombinant c-Abl and aliquots of the reaction mixture were analyzed by fluorescence spectroscopy and western blotting at ˜1 min and 60 min time-points. In the absence of ATP, essentially no change in FRET (i.e. the ratio of the fluorescein/tetramethylrhodamine emission intensities) was observed during the reaction (FIG. 3A), and no Rh-(Crk-II)-Fl phosphorylation could be detected using an anti-phosphotyrosine monoclonal antibody (FIG. 3B). In contrast, when ATP was included in the reaction mixture, a phosphorylation-dependent increase in the emission intensity ratio (a decrease in FRET) was consistently observed. Rh-(Crk-II)-Fl was completely phosphorylated after 1 h as determined by native PAGE mobility (FIG. 3C). The quite modest decrease in FRET (˜3% after 60 min) suggests that the SH2-phosphotyrosine interaction, which is triggered by Rh-(Crk-II)-Fl phosphorylation, results in only a small net change in the relative distance between the - and C-termini in the protein.

An interaction between the central SH3 domain of Crk-II (N-SH3) and a proline-rich region in c-Abl (located C-terminal to its kinase domain) has been implicated in formation of the enzyme-substrate complex. Mutations in either this proline rich region or in the N-SH3 domain, which are predicted to disrupt this intermolecular association, lead to impaired phosphorylation of Crk derivatives (24, 25, 27). Similarly, an interaction between the SH2 domain of Crk and the SH3 domain of c-Abl may also contribute to formation of the complex (28). A truncated version of c-Abl lacking this proline rich region and the SH3 domain would not be expected to phosphorylate Crk-II with normal kinetics. Indeed, treatment of Rh-(Crk-II)-Fl with a recombinant c-Abl fusion consisting of only the SH2 and kinase domains, did not lead to any detectable phosphorylation over 60 min as indicated by fluorescence and western blotting analysis (FIG. 4A). Note, an optimized peptide substrate (EAIYAAPFAKKK (SEQ ID No:2)(20)) was completely phosphorylated by this truncated version of the kinase after 60 min.

Taken together, the above studies indicate that Rh-(Crk-II)-Fl is a fluorescence biosensor for c-Abl phosphorylation of Crk-II and confirm that regions of c-Abl out with the SH2 and kinase domains are crucial for this process. One potential use for this biosensor is in the rapid screening of c-Abl kinase inhibitors or compounds that inhibit interactions necessary for phosphorylation. As a simple illustration, in an Example below, the system was used to investigate whether an exogenous ligand for the N-SH3 of Crk-II can modulate Crk-II phosphorylation by inhibiting binding to c-Abl. Treatment of Rh-(Crk-II)-Fl with full length c-Abl in the presence of a saturating amount of a high affinity N-SH3 ligand (21), resulted in a ˜50% reduction in the change in FRET after 60 minutes reaction, relative to the positive control (FIG. 4B). This suggests that the peptide ligand interferes with but does not completely inhibit phosphorylation, a conclusion substantiated by western blotting analysis (FIG. 4B).

Other methods may be used to screen for modulators of activity using the constructs and methods of the invention. In the example of phosphorylation described herein, radiolabeled ³²P substrates are not necessary to identify modulators of phosphorylation targets. Any polypeptide which undergoes a conformational change which can be reported by the insertion of two sensor peptides therein is a candidate for the methods of the present invention.

A solid-phase protein ligation procedure is described which allows a series of polypeptide fragments to be assembled in a manner analogous to SPPS. Importantly, the functionality's necessary for chemical ligation, N-terminal protection and attachment to the solid-support are readily incorporated into both recombinant and synthetic polypeptides. Thus, a combination of synthetic and recombinant polypeptide building blocks can be used in the procedure.

As illustrated in FIG. 2A, SPPL was used to prepare a dual-labeled version of Crk-II from three fragments; full length recombinant Crk-II and two small synthetic peptides. The well-established native chemical ligation reaction (6) was used to hook the polypeptides together in a stepwise fashion. In each of the two ligation reactions, a large excess of the synthetic component (>10 equivalents) was added to drive the reaction to completion. The first ligation, between the Crk-II-intein fusion and Cys-Fl-PS-Biotin, was performed directly from the chitin affinity beads, and was most efficient when both ethanethiol and MESNA were included as thiol cofactors. Ethanethiol has previously been shown to cleave intein-fusions more efficiently than MESNA (29). It is thus likely that Crk-II is cleaved off the chitin beads predominantly as an ethyl ^{α}thioester derivative and that this is then converted through transthioesterification into a more reactive MESNA ^{α}thioester derivative in situ. The second ligation reaction was performed on the solid-phase and thus the excess peptide was simply removed from the resin-bound product by washing (a gel filtration step was required after the first ligation).

Attachment to the solid-phase was achieved through a biotin-monomeric avidin interaction (note, in many cases it will be possible to directly introduce a biotin group at the C-terminus of the recombinant polypeptide (30)). This association was stable to the reducing conditions of ligation and was not disrupted by the ‘factor Xa’ deprotection step. It was also stable to a combination of high salt and detergent at pH 5.2 and pH 8.0, which permitted stringent washing of the column, importantly, allowing removal of trace amounts of bacterial protease contaminants which had been carried through from Crk-II protein expression. Upon completion of the synthesis, the semi-synthetic protein was eluted from the support by washing with exogenous biotin. Note that in order to maximize the recovery of the protein, this competitive elution procedure may have to be repeated several times. Alternatively, a proteolytic cleavage strategy could be employed which took advantage of the recognition sequence for the PreScission protease, incorporated between the biotin and fluorescein moieties in the C-terminal peptide. This latter strategy allows the use of higher capacity tetrameric avidin beads, although in some systems it may be less specific than competitive elution with biotin.

Factor Xa induced deprotection of the immobilized intermediate, Xa-Cys-(Crk-II)-Fl-PS-Biotin, proceeded efficiently and was complete after 3 h. However, a small amount of non-specific cleavage was observed, leading to an unreactive lower molecular weight fragment. This was easily removed by gel filtration post assembly—conceivably such side-products could also be removed using an orthogonal N-terminal affinity purification strategy. It is also worth noting that the use of alternative proteolytic deprotection strategies, based on enzymes such as enterokinase or ubiquitin hydrolase, may lead to less non-specific cleavage than factor Xa in certain protein systems.

SPPL has allowed the synthesis of a semi-synthetic Crk-II analog in which the FRET pair, Rh and Fl, were specifically introduced at the - and C-termini of the protein. The two fluorophores were positioned close to the natural ends of Crk-II (≦10 Å) in order to maximize the sensitivity to conformational change in this region. This type of chemical-labeling is analogous to the incorporation of different GFP derivatives at the termini of recombinant proteins through standard DNA cloning methodologies (31, 32,33).

Rh-(Crk-II)-Fl was found to biosense for c-Abl phosphorylation of Crk-II. Treatment with the full length kinase induced a small but reproducible decrease in FRET between the two fluorophores which was dependent upon phosphorylation as indicated by western blotting. Although western analysis was crucial to the initial validation of the approach, it should be stressed that FRET provides a direct (i.e. more rapid) and quantitative readout of Crk-II phosphorylation and hence c-Abl kinase activity. From a theoretical standpoint, which Applicants have non duty to disclose or be bound by, the results herein argue that the distance between the termini of Crk-II slightly increases after this post-translational event, implying that there is either a gross re-organization of the termini which results in only a small net distance change or that the conformational changes are remote from the termini.

The resonance energy transfer between the fluorophores in the unphosphorylated molecule was calculated to be 52.5% as determined from both the quenching of the fluorescein emission intensity and the sensitized emission of the rhodamine acceptor (as in ref. 34). Assuming that both fluorophores have random orientations and using a Forster distance of 45 Å for the Fl-Rh pair (34), then the distance between the two fluorophores is ˜44 Å. Interestingly, this suggests that unphosphorylated Crk-II has a somewhat compacted domain architecture, as opposed to a linear array of domains; based on the primary sequence, the - and C-termini could be as much as ˜200 Å apart if the inter-domain linkers assume a fully extended conformation.

The present invention also extends to the use of the multiple labeled constructs of the invention in identifying distances between interacting groups on target polypeptides.

A truncated version of c-Abl lacking both the proline-rich C-terminal region and the SH3 domain does not induce a FRET change in Rh-(Crk-II)-Fl which, as expected, is due to a complete lack of phosphorylation of this protein over the time-frame of the experiment. This both substantiates the ability of the Crk-II analog to specifically biosense phosphorylation and confirms that regions out with these domains are crucial for this process. It also indicates how such a biosensor maybe used for assaying the kinase activity of c-Abl or exploring the molecular mechanisms of Crk-II phosphorylation.

The deregulation of protein tyrosine kinases, such as c-Abl, has been implicated in the development of many disease states, making these proteins important targets in the drug discovery field (35). Current approaches for screening small molecule inhibitors mostly rely on the use of ³²P phospho-transfer assays, which are both expensive and create obvious safety issues. Non-radioactive assays that enable compounds to be rapidly screened are thus of significant value. In principle, the fluorescence-based strategy of the invention can be used for this purpose. As a simple demonstration, the system herein was used to rapidly assay the effect of a high affinity ligand for the N-SH3 of Crk on phosphorylation by c-Abl. This compound was found to partially inhibit Crk-II phosphorylation, presumably by blocking crucial interactions with the proline rich region of c-Abl.

The present invention may be better understood by reference to the following non-limiting Examples, which are provided as exemplary of the invention. The following examples are presented in order to more fully illustrate the preferred embodiments of the invention. They should in no way be construed, however, as limiting the broad scope of the invention.

### EXAMPLE I

### Preparation of Dual-Labeled Crk-II

Protein Expression; Xa-Cys-(Crk-II)-Intein-CBD: The polymerase chain reaction (5′ primer AAA AGA AAA AAA GGC GGC CGC TCG GAT CTG ATC GAA GGT CGT TGT GCG GGC AAC TTC GAC TCG G (SEQ ID No:3) and 3′ primer GCA AAC TGG CTC TTC CGC AGC CGC TGA AGT CCT CAT CGG G (SEQ ID No:4)), was used to amplify the region corresponding to full length mouse Crk-II (residues A2 to S304) from a pcDNA-mCrk vector template. After digestion with Sap1 and Not1, the desired fragment was purified by gel electrophoresis and subcloned into a Sap1-Not1 treated pTYB3 plasmid (New England Biolabs). This pTYB3_{Xa-Cys-Crk-II} vector encodes a fusion protein consisting of full length mouse Crk-II linked via a glycine residue to the N-terminus of the yeast VMA intein-CBD region and containing the sequence MASSRVDGGRSDLIEGRC (SEQ ID No:5) immediately N-terminal to Ala2 of Crk-II (confirmed by DNA sequencing): The pro-sequence up to but not including the Cys residue is hereafter referred to as ‘Xa-’ or -IEGR-. E coli BL21 cells were transformed with this plasmid and grown in LB medium (6 L) to mid-log phase. Protein expression was then induced for 4 h at 30° C. using 0.2 mM IPTG. After centrifugation the cells were resuspended in lysis buffer (0.1 mM EDTA, 250 mM NaCl, 5% glycerol, 1 mM PMSF, 25 mM HEPES, pH 7.4) and lysed using a French press. The soluble fraction was then loaded onto a chitin column (˜12 mL), pre-equilibrated in wash buffer (1 mM EDTA, 250 mM NaCl, 0.1% Triton X-100, 25 mM HEPES, pH 7.0) which was then washed with the same buffer. Typically, this procedure gave a loading of ˜2 mg fusion protein per mL chitin beads.

Peptide Synthesis: Peptides were manually synthesized according to the in situ neutralization/HBTU activation protocol for Boc-SPPS (5). The peptide, N-Tetramethylrhodamine-KRG-propionamide ^{α}thioester, was assembled on a S-propionamide derivatized 4-methylbenzhydrylamine (MBHA) resin (7), whereas CGK-[Dapa(Fl)]-GLEVLFQGPVRKG-[K^{ε}-(Biotin)]-G-NH₂ (Cys-Fl-PS-Biotin) (SEQ ID No:6) was synthesized using an MBHA resin. Orthogonal NH₂ protection allowed direct solid-phase attachment of the tetramethylrhodamine (Rh), fluorescein (Fl) and biotin groups which were each activated as the corresponding NHS-ester.

Solid-Phase Protein Ligation: [Rh-(Crk-II)-Fl], was prepared as follows: Note; All the steps are performed in the dark and at 4° C. unless otherwise stated.

Step 1—Loading: Purified Cys-Fl-PS-Biotin peptide (1 mM) was dissolved in ligation buffer (0.1% Triton-X 100, 200 mM NaCl, 200 mM phosphate pH 7.3) containing both 2-mercaptoethanesulfonic acid (MESNA 4% w/v) and ethanethiol (3% v/v) and then added to the pre-equilibrated chitin beads containing immobilized Xa-Cys-(Crk-II)-Intein-CBD (5 mL), to give a 50% slurry. The mixture was rocked for 48 h at room temperature at which time>95% of the protein (as determined by SDS-PAGE) had reacted to form the desired ligation product [Xa-Cys-(Crk-II)-Fl-PS-Biotin]: ESMS; observed mass=38,010±19 Da, expected (av. isotope comp.) 38,027 Da. DTT was then added to the ligation mix to give a 10 mM final concentration and the excess unreacted peptide removed by gel filtration (HR-75 column; running buffer, 0.1% Triton X-100, 2 mM DTT, 140 mM NaCl, 50 mM Tris, pH 7.4). A portion of the isolated ligation product (typically 1-2 mg) was then incubated for 1 h at 4° C. with 4 mL of monomeric avidin beads (Pierce) which had been pre-equilibrated in gel column buffer. Unbound contaminants were then removed by washing the beads with wash buffer A (0.2% Triton X-100, 2.5 mM DTT, 400 mM NaCl, 100 mM sodium acetate buffer, pH 5.2) followed by wash buffer B (0.2% Triton X-100, 2.5 mM DTT, 400 mM NaCl, 50 mM Tris, pH 8.0), 20 column volumes each. This gave a final loading of ˜0.4 mg of [Xa-Cys-(Crk-II)-Fl-PS-Biotin] per mL of monomeric avidin beads.

Step 2—Deprotection. The monomeric avidin beads were equilibrated into deprotection buffer (140 mM NaCl, 5 mM phosphate, pH 7.3) and then treated with factor Xa (10 U/mL of beads) for 3 h at room temperature. This facilitated complete removal of the cysteine protecting pro-sequence (Xa) as determined by SDS-PAGE, to generate the desired material containing a free N-terminal cysteine [Cys-(Crk-II)-Fl-PS-Biotin]: ESMS; observed mass=36,370±18 Da, expected (av. isotope comp.) 36,369 Da. The beads were then washed thoroughly with wash buffer C (5 mM DTT, 140 mM NaCl, 5 mM phosphate, pH 7.2) to remove the protease.

Step 3—Ligation: The beads were equilibrated into ligation buffer and a solution of purified Rh-KRG-propionamidethioester peptide with MESNA in ligation buffer added to give a 50% slurry of beads containing 2% w/v MESNA and ˜2.5 mM synthetic peptide. After rocking the mixture overnight, all of the protein had reacted (as determined by SDS-PAGE) forming the desired ligation product [Rh-(Crk-II)-Fl]. Unreacted peptide was removed by washing with ligation buffer and recycled; the beads were then further washed with ligation buffer supplemented with 2 mM DTT.

Step 4—Cleavage: The beads were washed with cleavage buffer (1 mM DTT, 0.1% Triton X-100, 1 mM EDTA, 140 mM NaCl, 50 mM Tris, pH 7.0, 20 column volumes) and the protein liberated from the monomeric avidin support by either; (i) competitive desorption or, (ii) proteolysis. (i) To compete the protein off of the monomeric avidin support, the beads were incubated with 8 column volumes of 2 mM biotin in cleavage buffer (˜55% of Rh-(Crk-II)-Fl was eluted in this step, though further material could be obtained on repeating the process). The supernatant was then passed over a gel filtration column as in Step 1 to obtain the desired pure material: ESMS; observed mass=37,132±18 Da, expected (av. isotope comp.) 37,124 Da. (ii) For proteolytic cleavage, the beads were treated overnight with 1 column volume of cleavage buffer containing the enzyme PreScission (Amersham Pharmacia, 2.5 U/mL of beads). The supernatant was then passed over a glutathione-agarose column to remove the protease and yield the desired material: ESMS; observed mass=36,125±18 Da, expected (av. isotope comp.) 36,118 Da.

The overall scheme for the synthesis of Rh-(Crk-II)-Fl by SPPL is summarized in FIG. 2A. As with SPPS, the strategy can be divided essentially into three parts; attachment of the first building block to a solid support, chain assembly in a C-to-N direction involving successive deprotection and ligation steps, and cleavage of the completed polypeptide off the solid support.

In the first step, full length mouse Crk-II was expressed as an in-frame fusion to an engineered yeast VMA intein which allows the subsequent generation of a reactive ^{α}thioester derivative of Crk-II. An extra Gly residue was added to the C-terminus of Crk-II to improve the kinetics of the first ligation reaction (8), and the N-terminal Met was replaced by the sequence -IEGRC (Xa-Cys) to facilitate controlled sequential ligation (17). Soluble expression of this fusion protein [Xa-Cys-(Crk-II)-Intein-CBD] was optimized using standard protocols (no in vivo intein cleavage of the full length fusion could be detected) and the desired material purified by affinity chromatography using a chitin column.

A synthetic peptide, Cys-Fl-PS-Biotin, containing both a fluorescein probe (Fl) and a biotin affinity handle separated by a linker region containing the cleavage site for the PreScission protease [LEVLFQGP, (PS)], was chemoselectively ligated to the C-terminus of recombinant Crk-II using EPL. This ligation reaction was found to be >95% complete after 48 h in the presence of a large excess of peptide and the thiol cofactors ethanethiol and MESNA. Gel filtration was used to separate the unreacted peptide from the desired ligation product which was then attached to monomeric-avidin beads via its biotin functionality. Preliminary model studies had established that the monomeric-avidin-biotin complex was stable to all the washing, deprotection and ligation steps used in SPPL, but that the interaction can be disrupted under mild conditions with exogenous biotin. Trace amounts of unreacted Crk-II protein and any remaining bacterial protein contaminants were then removed by vigorously washing the beads with high salt and detergent at pH 5.2 and pH 8.0. This yielded the pure protein, Xa-Cys-(Crk-II)-Fl-PS-Biotin, immobilized on a solid-support (FIG. 2B, Lane 2).

In order to continue the solid-phase synthesis, the Xa pro-sequence must be removed from the immobilized Xa-Cys-(Crk-II)-Fl-PS-Biotin to give an N-terminal Cys residue ready for ligation to the next peptide fragment. (The Xa motif acts as an Na protecting group for the Cys residue in Crk-II and prevents uncontrolled self-ligation during the first ligation step (17)). Complete enzymatic deprotection was achieved by treatment of the beads with the protease, factor Xa, for 3 hours to give Cys-(Crk-II)-Fl-PS-Biotin (FIG. 2B, Lane 3). A small amount (˜10%) of a lower molecular weight protein contaminant was also observed (FIG. 2B, Lane 3, weak band 26 kDa) suggesting that some non-specific cleavage had occurred during this step. The proteolysis reaction was terminated by simply washing the protease from the column; DTT was included in this buffer to simultaneously reduce any disulfide bonds that may have formed during the deprotection step. The beads were then equilibrated into ligation buffer, and the newly exposed N-terminal cysteine residue reacted with a tetramethylrhodamine containing ^{α}thioester peptide (Rh-KRG-propionamide ^{α}thioester) in a second ligation step. A large excess of synthetic peptide was again used in the reaction and MESNA was added as the sole thiol cofactor. This reaction was deemed complete after overnight incubation, as determined by SDS-PAGE analysis of the beads (FIG. 2B, Lane 4), generating the dual-labeled Crk-II derivative, Rh-(Crk-II)-Fl. The beads was then thoroughly washed to remove all unreacted tetramethylrhodamine peptide.

Rh-(Crk-II)-Fl was desorbed from the solid support by washing the beads with a solution containing 2 mM biotin. Approximately 55% of the immobilized material was recovered in a single washing step, though further protein could be eluted by repeating this procedure. The combined washes were passed over a gel filtration column to remove the free biotin and to remove the protein contaminant arising from non-specific factor Xa proteolysis. The so-purified dual-labeled Crk-II analog was characterized by electrospray mass spectrometry (FIG. 2C) and fluorescence spectroscopy (FIG. 2D), and was shown to bind a phosphotyrosine column and a peptide ligand specific to the central SH3 domain of Crk-II (data not shown), indicating that it had the same gross functional properties as the wild-type protein.

Note, we have also used tetrameric-avidin as the solid support for SPPL. However, due to the high affinity of this interaction, the completed protein cannot be competitively eluted from the column as above. In this case the beads were treated with the highly specific PreScission protease. The enzyme cleaved the construct at its recognition site, incorporated between the fluorescein and the biotinyl functionality's, releasing Rh-(Crk-II)-FI from the beads (data not shown).

### EXAMPLE II

### Kinase Assays

Purified Rh-(Crk-II)-Fl prepared as described in Example I was treated with either full length recombinant (Baculovirus/SF9) mouse c-Abl or a GST fusion of mouse c-Abl containing only the SH2 and kinase domains (expressed in E. coli BL21 essentially as described (19)). In a typical experiment, the appropriate c-Abl construct was incubated in reaction buffer (2 mM DTT, 0.2 mg/ml BSA, 10 mM Mg²⁺, 50 mM Tris, pH 7.4, either with or without ATP (500 μM)) for 5 min at 30° C. before addition of Rh-(Crk-II)-Fl (final concentration=0.25 μM). In order to ensure that equal amounts of active c-Abl enzyme were added to each reaction, preliminary titration experiments were carried out using an optimized peptide substrate for c-Abl (EAIYAAPFAKKK (SEQ ID No:2) (20)). For peptide inhibition studies, Rh-(Crk-II)-Fl was pre-incubated for 30 min with a high affinity ligand for the N-SH3 domain of Crk, PPPALPPKRRR-NH₂ (SEQ ID No:7) (21), such that the final concentration of ligand in the kinase assay was 12 μM. In all cases, aliquots of the reaction mixtures were removed at ˜1 min and 60 min, quenched with EDTA (final conc.=40 mM), and then analyzed by native-PAGE and/or Western blotting and fluorescence spectroscopy.

Western Blotting. Standard procedures were used to probe for tyrosine phosphorylation using a mouse monoclonal anti-phosphotyrosine primary antibody (PY20, Santa Cruz Biotechnology) and a HPO-conjugated goat anti-mouse polyclonal secondary antibody (Amersham Pharmacia).

Fluorescence Spectroscopy. Experiments were conducted at 18° C. in a stirred 0.5 cm-pathlength cell using a SPEX FL3-11C fluorimeter. Samples from the reactions (50 μl) were diluted into 2 mM DTT, 0.4 mg/mL BSA, 140 mM NaCl, 50 mM Tris, pH 7.4 buffer (450 μl) for analysis. Excitation was at 490 nm with a 2.5 nm slit and the fluorescence emission was monitored at 520 nm and 580 nm through a 4 nm slit. Purified Rh-(Crk-II)-Fl was assayed for its ability to biosense Crk-II phosphorylation by the c-Abl protein tyrosine kinase. As indicated previously, phosphorylation by c-Abl leads to an intramolecular association between a phosphotyrosine motif and the Crk-II SH2 domain, which could potentially be reported by the dual-labeled Crk-II derivative (FIG. 1). Rh-(Crk-II)-Fl was treated with full length recombinant c-Abl and aliquots of the reaction mixture were analyzed by fluorescence spectroscopy and western blotting at ˜1 min and 60 min time-points. In the absence of ATP, essentially no change in FRET (i.e. the ratio of the fluorescein/tetramethylrhodamine emission intensities) was observed during the reaction (FIG. 3A), and no Rh-(Crk-II)-Fl phosphorylation could be detected using an anti-phosphotyrosine monoclonal antibody (FIG. 3B). In contrast, when ATP was included in the reaction mixture, a phosphorylation-dependent increase in the emission intensity ratio (a decrease in FRET) was consistently observed. Rh-(Crk-II)-Fl was completely phosphorylated after 1 h as determined by native PAGE mobility (FIG. 3C). The quite modest decrease in FRET (˜3% after 60 min) suggests that the SH2-phosphotyrosine interaction, which is triggered by Rh-(Crk-II)-Fl phosphorylation, results in only a small net change in the relative distance between the - and C-termini in the protein.

An interaction between the central SH3 domain of Crk-II (N-SH3) and a proline-rich region in c-Abl (located C-terminal to its kinase domain) has been implicated in formation of the enzyme-substrate complex. Mutations in either this proline rich region or in the N-SH3 domain, which are predicted to disrupt this intermolecular association, lead to impaired phosphorylation of Crk derivatives (24, 25, 27). Similarly, an interaction between the SH2 domain of Crk and the SH3 domain of c-Abl may also contribute to formation of the complex (28). A truncated version of c-Abl lacking this proline rich region and the SH3 domain would not be expected to phosphorylate Crk-II with normal kinetics. Indeed, treatment of Rh-(Crk-II)-Fl with a recombinant c-Abl fusion consisting of only the SH2 and kinase domains, did not lead to any detectable phosphorylation over 60 min as indicated by fluorescence and western blotting analysis (FIG. 4A). Note, an optimized peptide substrate (EAIYAAPFAKKK (20)) was completely phosphorylated by this truncated version of the kinase after 60 min (data not shown).

Taken together, the above studies indicate that Rh-(Crk-II)-Fl is a fluorescence biosensor for c-Abl phosphorylation of Crk-II and confirm that regions of c-Abl out with the SH2 and kinase domains are crucial for this process. One potential use for this biosensor is in the rapid screening of c-Abl kinase inhibitors or compounds that inhibit interactions necessary for phosphorylation. As a simple illustration, the system was used to investigate whether an exogenous ligand for the N-SH3 of Crk-II can modulate Crk-II phosphorylation by inhibiting binding to c-Abl. Treatment of Rh-(Crk-II)-Fl with full length c-Abl in the presence of a saturating amount of a high affinity N-SH3 ligand (21), resulted in a 50% reduction in the change in FRET after 60 minutes reaction, relative to the positive control (FIG. 4B). This suggests that the peptide ligand interferes with but does not completely inhibit phosphorylation, a conclusion substantiated by western blotting analysis (FIG. 4B).

The present invention is not to be limited in scope by the specific embodiments describe herein. Indeed, various modifications of the invention in addition to those described herein will become apparent to those skilled in the art from the foregoing description and the accompanying figures. Such modifications are intended to fall within the scope of the appended claims.

Various publications are cited herein, the disclosures of which are incorporated by reference in their entireties.
1. Cornish, V. W., Mendel, D. & Schultz, P. G. (1995) Angew. Chem. Int. Ed. Engl. 34, 621-633.
2. Chen, Y., Ebright, Y. W. & Ebright, R. H. (1994) Science 265, 90-92.
3. Wilken, J. & Kent, S. B. H. (1998) Curr. Opin. Biotech. 9, 412-426.
4. Jackson, D. Y., Burnier, J., Quan, C., Stanley, M., Tom, J. & Wells, J. A. (1994) Science 266, 243-247.
5. Schnölzer, M., Alewood, P., Jones, A., Alewood, D. & Kent, S. B. H. (1992) Int. J. Pept. Protein Res. 40, 180-193.
6. Dawson, P. E., Muir, T. W., Clark-Lewis, I. & Kent, S. B. H. (1994) Science 266, 776-779.
7. Camarero, J. A., Cotton, G. J., Adeva, A. & Muir, T. W. (1998) J Pep. Res. 51, 303-316.
8. Hackeng, T. M., Griffin, J. H. & Dawson, P. E. (1999) Proc. Natl. Acad. Sci. USA 96, 10068-10073.
9. Canne, L. E., Botti, P., Simon, R. J., Chen, Y., Dennis, E. A. & Kent, S. B. H. (1999) J. Am. Chem. Soc. 121, 8720-8727.
10. Erlanson, D. A., Chytil, M. & Verdine, G. L. (1996) Chem. Biol. 3, 981-991.
11. Chong, S., Mersha, F. B., Comb, D. G., Scott, M. E., Landry, D., Vence, L. M., Perler, F. B., Benner, J., Kucera, R. B., Hirvonen, C. A., et al. (1997) Gene 192, 271-281.
12. Mathys, S., Evans Jr, T. C., Chute, C. I., Wu, H., Chong, S., Benner, J., Liu, X.-Q. & Xu, M.-Q. (1999) Gene 231, 1-13.
13. Southworth, M. W., Amaya, K., Evans, T. C., Xu, M.-Q. & Perler, F. B. (1999) BioTechniques 27, 110-120.
14. Muir, T. W., Sondhi, D. & Cole, P. A. (1998) Proc. Natl. Acad. Sci. USA 95, 6705-6710.
15. Evans Jr., T. C., Benner, J. & Xu, M.-Q. (1998) Protein Science 7, 2256-2264.
16. Cotton, G. J. & Muir, T. W. (1999) Chem. Biol. 6, R247-R256.
17. Cotton, G., Ayers, B., Xu, R. & Muir, T. W. (1999) J. Am. Chem. Soc. 121, 1100-1101.
18. Merrifield, R. B. (1963) J. Am. Chem. Soc. 85, 2149-2154.
19. Garcia, P., Shoelson, S. E., George, S. T., Hinds, D. A., Goldberg, A. R. & Miller, W. T. (1993) J. Biol. Chem. 33, 25146-25151.
20. Songyang, Z., Carraway III, K. L., Eck, M. J., Harrison, S. C., Feldman, R. A., Mohammadi, M., Schlessinger, J., Hubbard, S. R., Smith, D. P., Eng, C., et al. (1995) Nature 373, 536-539.
21. Posern, G., Zheng, J., Knudsen, B. S., Kardinal, C., Muller, K. B., Voss, J., Shishido, T., Cowburn, D., Cheng, G., Wang, B., et al. (1998) Oncogene 16, 1903-1912.
22. Birge, R. B., Knudsen, B. S., Besser, D. & Hanafusa, H. (1996) Genes to Cells 1, 595-613.
23. Buday, L. (1999) Biochim. Biophys. Acta 1422, 187-204.
24. Feller, S. M., Knudsen, B. & Hanafusa, H. (1994) EMBO J. 13, 2341-2351.
25. Hashimoto, Y., Katamaya, H., Kiyokawa, E., Ota, S., Kurata, T., Gotoh, N., Otsuka, N., Shibata, M. & Matsuda, M. (1998) J. Biol. Chem. 273, 17186-17191.
26. Rosen, M. K., Yamazaki, T., Gish, G. D., Kay, C. M., Pawson, T. & Kay, L. E. (1995) Nature 374,477-479.
27. Ren, R., Ye, Z.-S. & Baltimore, D. (1994) Genes Dev. 8, 783-795.
28. Anafi, M., Rosen, M. K., Gish, G. D., Kay, L. E. & Pawson, T. (1996) J. Biol. Chem. 35, 21365-21374.
29. Ayers, B., Blaschke, U. K., Camarero, J. A., Cotton, G. J., Holford, M. & Muir, T. W. (1999) Biopolymers 51, in press.
30. Smith, P. A., Tripp, B. C., DiBlasio-Smith, E. A., Lu, Z., LaVallie, E. R. & McCoy, J. M. (1998) Nucleic Acids Res. 26, 1414-20.
31. Tsien, R. Y. (1998) Annu. Rev. Biochem. 67, 509-544.
32. Miyawaki, A., Llopis, J., Heim, R., McCaffery, J. M., Adams, J. A., Ikura, M. & Tsien, R. Y. (1997) Nature 388, 882-886.
33. Romoser, V. A., Hinkle, P. M. & Persechini, A. (1997) J. Biol. Chem. 272, 13270-13274.
34. Selvin, P. R. (1995) Methods Enzymol. 246, 300-334.
35. Cohen, P. (1999) Curr. Opin. Chem. Biol. 3, 459-465.
36. Collaci, S., Heyduk, E., & Heyduk, T. (1999) Mol. Cell 3, 229-238.

1-50. (canceled)

51. A composition for detecting the effect of an enzyme on a peptide substrate, the activity of said enzyme being effective to convert said peptide substrate, w/o cleavage, from an unmodified state to a modified state, said composition comprising a functional peptide substrate for said enzyme, having a first detectable proximity-sensor peptide incorporated into a first position of said substrate and a second detectable proximity-sensor peptide incorporated into a second position of said substrate, thereby providing a semi-synthetic multiple labeled polypeptide substrate having a first structural conformation in said unmodified state and a second structural conformation in said modified state, said proximity sensors being spaced apart in said first structural conformation at a distance which is characteristic of said unmodified state and being spaced apart in said second structural conformation at a distance which is characteristic of said modified state, detection of one of said structural conformations being indicative of the effect of said enzyme on said substrate.

52. The composition of claim 51, wherein said enzyme is a kinase.

53. The composition of claim 52, wherein said kinase is Abelson protein tyrosine kinase.

54. The composition of claim 51, wherein said peptide substrate is Crk-II.

55. The composition of claim 51, wherein said modification of said substrate is a post-translational type modification.

56. The composition of claim 55, wherein said modification of said substrate is a phosphorylation modification.

57. The composition of claim 55, wherein said modification of said substrate is a dephosphorylation modification.

58. The composition of claim 51, further comprising a modulator of said enzyme.

59. The composition of claim 58, wherein said modulator of said enzyme inhibits said enzyme activity.

60. The composition of claim 58, wherein said modulator of said enzyme activates said enzyme activity.

61. The composition of claim 51 wherein said first detectable proximity-sensor peptide is at the N-terminus, the C-terminus of which is peptide-bonded to the N-terminus of said semi-synthetic multiple labeled polypeptide, the C-terminus of which is peptide bonded to the N-terminus of said second detectable proximity-sensor peptide.

62. The composition of claim 51 wherein said peptide substrate is a recombinant polypeptide.

63. The composition of claim 51 wherein said first and second detectable proximity-sensor peptides of said semi-synthetic multiple labeled polypeptide comprise a FRET pair.

64. The composition of claim 63 wherein said FRET pair is selected from the group consisting of fluorescein and tetramethylrhodamine, IAEDANS and fluorescein, EDANS and DABCYL, BODIPY fluorescein and BODIPY FL fluorescein, β-phycoerythrin and CY5, and pyrene and coumarin.

65. The composition of claim 63, wherein said FRET pair comprises fluorescein and tetramethylrhodamine.

66. The composition of claim 51 wherein said detectable proximity-sensor peptide is a synthetic oligopeptide comprising a fluorescent amino acid derivative.

67. The composition of claim 51 as set forth in FIG. 5A (SEQ ID NO: 8).

68. The composition as shown in SEQ ID NO: 9.
